(19) United States
(12) Patent Application Publication
(10) Pub. No.: US 2024/0206717 A1
(43) Pub. Date: Jun. 27, 2024

(54) ILLUMINATION SOURCES AND METHOD FOR DENTAL TRANSILLUMINATION

(71) Applicant: Attent AS, Lillesand (NO)

(72) Inventor: Kjell MYKLAND, Lillesand (NO)

(21) Appl. No.: 18/570,893

(22) PCT Filed: Nov. 25, 2021
(86) PCT No.: PCT/EP2021/083022

§ 371 Date: Dec. 15, 2023

(30) Foreign Application Priority Data
Nov. 25, 2020 (GB) 2018542.7

(51) Int. Cl. A61B 1/06; A61B 1/24; A61C 9/00

(52) U.S. Cl. CPC A61B 1/0625; A61B 1/0655; A61B 1/24; A61C 9/0053

(57) ABSTRACT

A head portion (112) for a device for transillumination of a tooth comprises: a central portion (114), arranged to receive light from the tooth for imaging an occlusal/incisal view of the tooth, and first and second flexible wing portions (116) extending from the central portion (14). The first flexible wing portion is configured to contact a first side of the tooth, and the second flexible wing portion is configured to contact a second side of the tooth, opposite to the first side. The first flexible wing portion comprises a first plurality of illumination sources for illuminating the tooth from the first side, and the second flexible wing portion comprises a second plurality of illumination sources for illuminating the tooth from the second side, and wherein the first and second plurality of illumination sources are controllable to produce multiple different lighting conditions.

The present disclosure relates to a dental transillumination device and system for home use. Such a system may be used by a person who is not a dental practitioner, to be able to identify dental issues (such as caries, for example) without needing to visit a dentist.

It is common for dental patients to feel anxiety associated with visiting a dentist. This can lead to people avoiding a visit to their dentist, thereby missing the opportunity to identify dental issues early, whilst they are easy to treat. In some areas, it is difficult to access dental care, for example because there is no dentist close by, or because the cost of visiting a dentist is prohibitive. It is therefore desirable to provide some means to monitor dental health at home. If dental issues are identified at home, this can provide the motivation for people to visit the dentist, especially if they would otherwise be reluctant to do so. On the other hand, if a dental patient is able to determine at home that there are no issues with their teeth, they can defer a trip to the dentist.

According to the Centres for Disease Control and Prevention, 91%% of adults aged 20 to 64 in the US have dental caries (commonly referred to as tooth decay or cavities). If caries is identified early, preventative measures can be suggested by a dentist in order to prevent further deterioration in the condition of the teeth. Without such preventative measures, the caries may worsen, necessitating more invasive treatments (such as drilling the tooth and filling the cavity).

Caries can be categorised visually by a dentist as one of category 1 to category 6 according to the International Caries Detection and Assessment System (ICDAS). The visual appearance associated with each code is set out below:
1—White/brown spot in dry enamel.
2—White/brown spot in wet enamel.
3—Micro-cavity in dry enamel <0.5 mm without visible dentin.
4—Dark dentine shadow seen through wet enamel with or without micro-cavity.
5—Dentin exposure in cavity >0.5 mm to half the dental surface.
6—Dentin exposure in cavity greater than half of the dental surface.

Caries categorised as code 3 or above should be seen by a dentist, for treatment and/or ongoing monitoring.

As well as visual examination, a method traditionally used by dentist in order to identify caries is the use of x-rays to produce a radiograph of the patient's teeth. However, since x-rays are ionising radiation, there is understandable reluctance to use x-ray imaging too frequently. Typically, even those at high risk of dental problems will not have x-rays taken more frequently than once every 6 months. X-ray imaging must of course be carried out by a dental professional, and cannot be done at home by a patient themselves.

Dental transillumination is another technique which also allows for the identification of caries. In this technique, a tooth is illuminated by a bright light source which illuminates from one side of the tooth or both sides, typically with near infra-red (NIR) light. The light passes through the tooth and the tooth is imaged in the occlusal/incisal direction (the direction toward the biting surface of the teeth). Due to the optical properties of caries tissue, caries appear as a dark shadow on the image. Transillumination facilitates detection of caries in real-time, and since the light used is non-ionising, it can be carried out frequently.

Existing transillumination devices are very expensive and unsuitable for home use. In particular, existing device require the expertise of a dentist to capture images of sufficient quality to identify caries. Typically, such devices comprise a head portion with two parallel flexible wing portions which fit snugly either side of a tooth to be imaged. Light is incident onto the tooth from one or both of the wing portions, and the flexibility of the wing portions should allow for the light source to be maintained close to the tooth surface. However, such devices have been found to have shortcomings—for example, it is common for images of canines and incisors to be overexposed (due to too much light entering the tooth) whereas images of molars may be underexposed (due to insufficient light entering the tooth). It therefore requires the expertise of a dentist to position the device in such a way that satisfactory images are captured.

In view of the foregoing, it is desirable to provide a dental transillumination system for home use which would allow a person which is not a dental practitioner to be able to identify dental issues without needing to visit a dentist.

According to a first aspect of the present invention, there is provided a head portion for a device for transillumination of a tooth, the head portion comprising: a central portion, arranged to receive light from the tooth for imaging an occlusal/incisal view of the tooth, and first and second flexible wing portions extending from the central portion, wherein the first flexible wing portion is configured to contact a first side of the tooth, and the second flexible wing portion is configured to contact a second side of the tooth, opposite to the first side, wherein the first flexible wing portion comprises a first plurality of illumination sources for illuminating the tooth from the first side, and the second flexible wing portion comprises a second plurality of illumination sources for illuminating the tooth from the second side, and wherein the first and second plurality of illumination sources are controllable to produce multiple different lighting conditions.

Here, different lighting conditions correspond to different combinations of illumination sources being turned on to illuminate the tooth.

The first and second sides of the tooth may be the facial and lingual/palatal sides of the tooth. Here, the facial side of the tooth is the labial surface (the side nearest the lips) or the buccal surface (the side nearest the cheeks)—depending on the tooth. The lingual/palatal surface of the tooth is the side of the tooth closest to the tongue or palate, respectively.

Whether the image is an occlusal view or incisal view of the tooth depends on the tooth—the incisal surface is the biting edge of the canines and incisors, while the occlusal surface is the biting edge the molars and premolars. So images of the canines and incisors are termed incisal views and images of the molars and premolars are termed occlusal views.

The multiple lighting conditions may differ from one another in terms of the total light intensity incident into the tooth, and/or the angle(s) from which light is incident into the tooth. The multiple lighting conditions may be achieved by controlling how many illumination sources are illuminating the tooth, and/or by controlling which of the illumination sources, each in a different position, are illuminating the tooth.

Each of the illumination sources (i.e. each of the illumination sources of the first and second plurality of illumination sources) may be independently controllable. That is, every one of the illumination sources present in the head portion may be controllable independent of the status of any of the other illumination sources.

The illumination sources of the first and second plurality of illumination sources may be controllable in pairs. The pairings may be pairs on the same flexible wing portion, or the pairs may each comprise one illumination source from one flexible wing portion, and one illumination source from the other flexible wing portion.

Each flexible wing portion may comprise two groups of two illumination sources. A first group may comprise two illumination sources located so as to be closer to the tip of the tooth when the head portion is positioned on a tooth, and a second group may comprise two illumination sources located so as to be further from the tip of the tooth when the head portion is positioned on a tooth. Each of the first and second group may be independently controllable of the other group, and corresponding first and second groups on the second wing portion are also independently controllable of each other and of the first and second groups on the first wing.

The illumination sources of the first and second plurality of illumination sources may be controllable in groups of greater than two (but less than the total number of illumination sources).

Optionally, the plurality of illumination sources are LEDs.

The plurality of illumination sources may be configured to emit near-IR light. Here, near-IR light is defined as light having a wavelength in the range of 780 nm to 3000 nm (in accordance with the ISO 20473 standard). The plurality of illumination sources may be configured to emit light having a wavelength of between 780 nm and 1000 nm.

In one example, the illumination sources are LEDs having a peak wavelength at approximately 850 nm.

Optionally, the plurality of illumination sources all have the same nominal spectral characteristics (including the same peak wavelength and peak width). Alternatively, one or more illumination sources could have different spectral characteristics from the others (with peak wavelengths still within the near-IR range) to allow for images to be captured under a plurality of near-IR wavelengths.

The first plurality of illumination sources may comprise four illumination sources, and/or the second plurality of illumination sources may comprise four illumination sources.

In the case that four illumination sources are provided on a flexible wing portion, the four illumination sources of the first plurality of illumination sources may be arranged at the corners of a notional quadrilateral, for example a square or rectangle or a rhombus or parallelogram, facing the side of the tooth.

On a flexible wing portion, two or more illumination sources may be located so as to be further from the tip of the tooth when the head portion is in place on a tooth, and two or more illumination sources may be located so as to be positioned closer to the tip of the tooth when the head portion is in place on a tooth. The aim is for at least one of the illumination sources to be located adjacent to the gum-line when in use, no matter which tooth is being imaged. Here, adjacent to the gum-line means close to, but slightly vertically offset from the gum-line, so that light is still directed into the tooth rather than into the gum. This is advantageous because (as discussed later) a camera captures images from the tip of the tooth (i.e. facing the biting surface of the tooth), so to image the greatest possible volume of the tooth, and in particular to capture as much as possible of the shadows indicative of caries, it is advantageous to have light entering the tooth from near the gum-line. If the tooth is illuminated far from the gum-line, the shadows closer to the gum-line will be hidden from the camera. It depends on the tooth as to which of the illumination sources is in the best position; on a molar in the lower jaw for example the best-positioned illumination source may be the one positioned lowermost down the tooth when the head portion is in place on a tooth. On an incisor or canine in the lower jaw, for example, that same illumination source may be located below the gum-line, in which case it is not best-positioned to illuminate the tooth. In such a case, the best positioned illumination source may be one located closest to the tip of the tooth when the head portion is in place on the tooth.

The illumination sources positioned closer to the tip of the tooth in use are optionally positioned at a distance of 0.5 to 3 mm (for example, 1 mm to 2 mm, and optionally 1.75 mm) from the illumination sources located so as to be further from the tip of the tooth in use, measured as a perpendicular distance between the closest edges of the respective illumination sources.

The two or more illumination sources located so as to be closer to the tip of the tooth may be spaced apart at intervals of 3 to 7 mm, for example 5 mm (giving a corresponding spacing across the lateral direction of the tooth), measured as a perpendicular distance between the closest edges of the illumination sources. The two or more illumination sources located so as to be closer to the tip of the tooth may be positioned so that they are located at the same height along the flexible wing portion (so that a notional line joining their centres is approximately parallel to the gum-line when the head portion is positioned on a tooth), or they may be slightly vertically offset from one another. They may be vertically offset by approximately 1 mm for example, measured as the perpendicular distance between a line joining their centres.

Similarly, the two or more illumination sources located so as to be positioned further from the tip of the tooth may be spaced apart at intervals of 3 to 7 mm, for example 5 mm (giving a corresponding spacing across the lateral direction of the tooth), measured as a perpendicular distance between the closest edges of the illumination sources. The two or more illumination sources located so as to be further from the tip of the tooth may be positioned so that they are located at the same height along the flexible wing portion (so that a notional line joining their centres is approximately parallel to the gum-line when the head portion is positioned on a tooth), or they may be slightly vertically offset from one another. They may be vertically offset by approximately 1 mm for example, measured as the perpendicular distance between a line joining their centres.

Where four illumination sources are provided, two of the four illumination sources may be located so as to be further from the tip of the tooth when the head portion is in place on a tooth, and two of the four illumination sources may be located so as to be positioned closer to the tip of the tooth when the head portion is in place on a tooth. The two illumination sources located so as to be closer to the tip of the tooth may be positioned so that they are located at the same height along the flexible wing portion (so that a notional line joining their centres is approximately parallel to the gum-line when the head portion is positioned on a tooth), or they may be slightly vertically offset from one another (for example, by 1 mm, measured as the perpendicular distance between a line joining their centres). The two or more illumination sources located so as to be further from the tip of the tooth may be positioned so that they are located at the same height along the flexible wing portion (so that a notional line joining their centres is approximately parallel to the gum-line when the head portion is positioned on a tooth), or they may be slightly vertically offset from one another (for example, by 1 mm, measured as the perpendicular distance between a line joining their centres).

As noted above, the illumination sources are controllable. At least the on/off status of the illumination sources may be controllable. Optionally, the intensity of illumination of the illumination sources may be controllable.

The illumination sources may be controllable in such a way as to illuminate the first and second sides of the tooth simultaneously.

The head portion may comprise a flex PCB on which the plurality of illumination sources are mounted. The flex PCB may comprise two wings (one for each flexible wing portion), and each wing may be mounted onto a more rigid projection extending from the central portion. Of course, the rigid projection may be still flexible enough to allow the flexible wing portion to flex, so that the head portion can be slid onto a tooth.

The flexible wing portions may be covered by a soft covering which both protects the flex PCB and its components, and protects the user's teeth from damage by the head portion.

Each of the first and second flexible wing portions may comprise a projection (for example formed by the soft covering) arranged to space apart the illumination sources from the tooth. This may allow for the light from the illumination sources to be more evenly spread, compared to the case where the illumination sources are closer to the tooth. When the LEDs are too close to the tooth, there may be an uneven spread of light, and a risk of partial overexposure in the captured images.

Each of the first and second flexible wing portions may comprise a projection for example formed by the soft covering) arranged to prevent or reduce light from the illumination sources directly falling into a window in the central portion.

The central portion may comprise a plurality of alignment projections (for example, formed by the soft covering) bracketing the first and second flexible wing portions and arranged substantially perpendicular to the first and second flexible wing portions.

The head portion may comprise a chip for controlling the illumination sources. The illumination sources may each be separately connected to the chip to allow for independent control of each illumination source by the chip.

The control of the illumination sources may be carried out without input from the user, i.e. control of the illumination sources may be carried out automatically.

The invention extends to a transillumination device comprising the head portion of the first aspect (optionally incorporating any of the optional features of the head portion as described in the foregoing).

The head portion may be removably attached to the transillumination device. This allows a plurality of users to use the same transillumination device, but for each user to have their own head portion. Alternatively, the head portion may be an integral part of the transillumination device.

The head portion may comprise an RFID tag or NFC tag. This may allow for identification of a head portion as belonging to a particular user.

The transillumination device may comprise a camera. The camera may be a CMOS or CCD camera, for example. The camera may be a colour camera, or a monochromatic camera. The camera may be capable of capturing still images, and/or video.

The transillumination device may comprise an optical assembly. The optical assembly may comprise a prism and one or more lenses. The prism may receive light from the tooth via a window in the central portion of the head portion, and may bend the light down a neck portion of the transillumination device. Further optical components (optionally including an aperture and three plano-convex lenses) in the optical assembly may shape the light beam and focus it onto the imaging area of the camera.

In an alternative arrangement, the camera may be positioned directly opposite the window in the central portion of the head portion, obviating the need for a prism. An aperture and a plano-convex lenses may be positioned between the window in the central portion of the head portion and the camera.

The transillumination device may comprise a controller for controlling the illumination sources.

The transillumination device may comprise a wireless transceiver.

According to a second aspect of the invention, there is provided a method of imaging a tooth by transillumination comprising: controlling a plurality of illumination sources to apply a first lighting condition to the tooth, and imaging the tooth under the first lighting condition to acquire a first image; and controlling the plurality of illumination sources to apply a second lighting condition to the tooth, and imaging the tooth under the second lighting condition to acquire a second image, wherein the second lighting condition is different to the first lighting condition.

Here, the first and second lighting conditions are lighting conditions which allow for transillumination of the tooth to acquire first and second transillumination images of the tooth.

Optionally, the method may comprise analysing the first and second images to determine if the first and second images meet predetermined criteria for further analysis.

A plurality of further lighting conditions may also be applied, to allow for a further plurality of transillumination images of the tooth to be acquired.

According to a third aspect of the invention, there is provided a method of imaging a tooth by transillumination comprising: controlling a plurality of illumination sources to apply a first lighting condition to the tooth, wherein the first lighting condition is applied on the basis of knowledge of the type of tooth that is being imaged, and imaging the tooth under the first lighting condition to acquire a first image.

Optionally, knowledge regarding the type of the tooth includes information about the size and/or location of the tooth, and/or whether the tooth is an incisor, canine, pre-molar or molar. A shape recognition algorithm may be used to determine the type of tooth.

Optionally, the method comprises analysing the first image to determine if the first image meets predetermined criteria for further analysis, and if not, controlling a plurality of illumination sources to apply a second lighting condition to the tooth, and imaging the tooth under the second lighting condition.

According to a fourth aspect of the invention, there is provided a method of imaging a tooth by transillumination comprising: controlling a plurality of illumination sources to apply a first lighting condition to the tooth, wherein the first lighting condition is pre-set based on a prior calibration process carried out by the user, and imaging the tooth under the first lighting condition to acquire a first image.

In the calibration process, a plurality of lighting conditions may be sequentially applied to the tooth, and an image may be acquired under each lighting condition. Then, the images are analysed to determine the highest quality image. The lighting condition under which the highest quality image was acquired is then pre-set as the first lighting condition.

The highest quality image may for example be one with minimal reflections from stray light, and/or an image with the correct exposure (not overexposed by putting too much light into the tooth, or underexposed by putting too little light into the tooth), and/or an image with appropriate brightness and contrast.

The following optional features apply to any of the second, third or fourth aspects of the invention (and may also be combined with optional features described above in respect of those aspects).

The foregoing methods may include the use of the head portion of the first aspect (optionally incorporating any of the optional features described above in respect of the first aspect) or the use of the transillumination device described above.

The method may include imaging the tooth while the head portion is held stationary on the tooth, or whilst the head portion is being moved across the surface of the tooth, for example in a sweeping motion. Such a sweeping motion may comprise smoothly moving slowly from tooth to tooth.

The predetermined criteria for further analysis may include one or more selected from: the positioning of the tooth within the image, the uniformity of lighting in the image, the brightness and the contrast in the image.

The methods may comprise controlling the on/off status of the illumination sources to change the lighting condition. The methods may comprise controlling the intensity of illumination of the illumination sources to change the lighting condition.

The methods may comprise capturing multiple images of a tooth under illumination by near-IR at different wavelengths. The method may comprise combining the multiple images of the same tooth to make a combined image. Each image of the multiple images in the combined image may be combined with the others with a weighting factor of between 0 and 1, where 0 means no contribution and 1 means total contribution.

The methods may comprise illuminating the first and second sides of the tooth simultaneously.

The methods may comprise illuminating only one of the first or second sides of the tooth at a time.

The methods may comprise controlling the plurality of illumination sources individually, in pairs, or in groups of greater than two (but less than the total number of illumination sources). For example, each flexible wing portion may comprise two groups of two illumination sources. A first group may comprise two illumination sources located so as to be closer to the tip of the tooth when the head portion is positioned on a tooth, and a second group may comprise two illumination sources located so as to be further from the tip of the tooth when the head portion is positioned on a tooth.

Certain preferred embodiments of the invention will now be described by way of example only and with reference to the accompanying drawings, in which:

FIG. 1 shows an exemplary transillumination device;

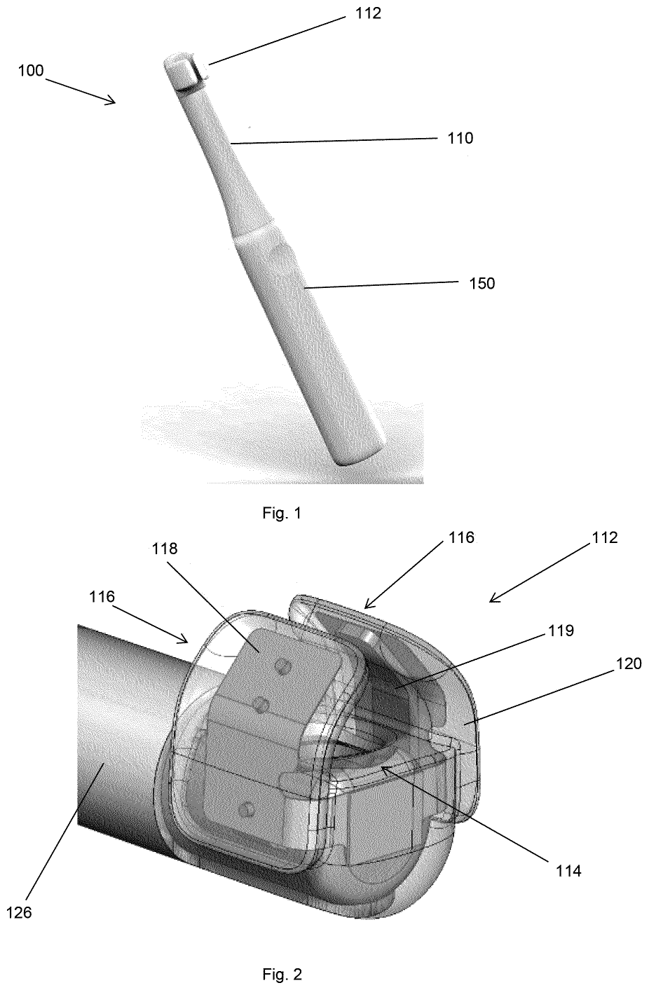

FIG. 2 shows a tip of the exemplary transillumination device;

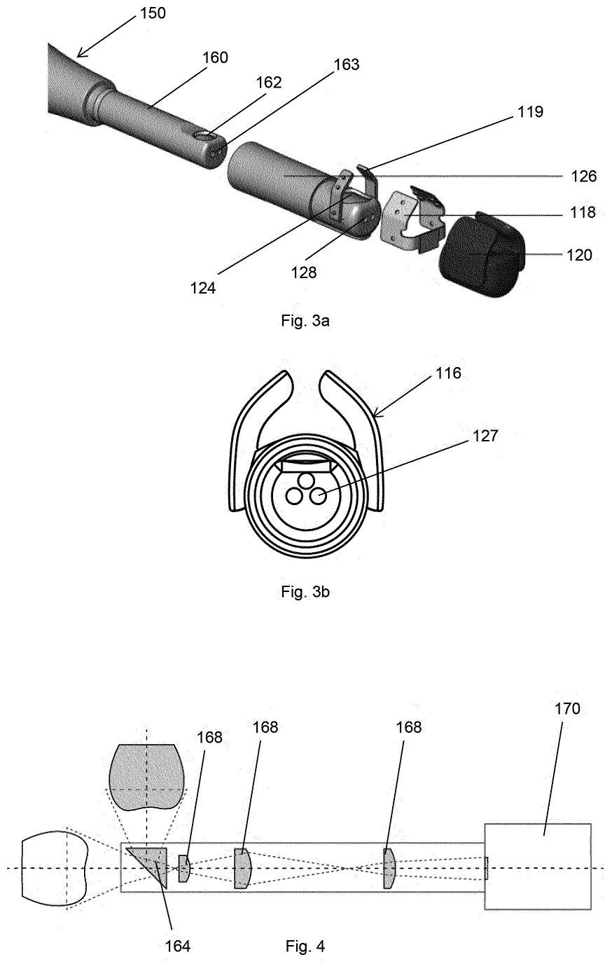

FIG. 3 shows an exploded view of the tip of the exemplary transillumination device, along with a neck portion of a main body portion of the exemplary transillumination device;

FIG. 4 shows an optical assembly within the neck portion of a main body portion of the exemplary transillumination device;

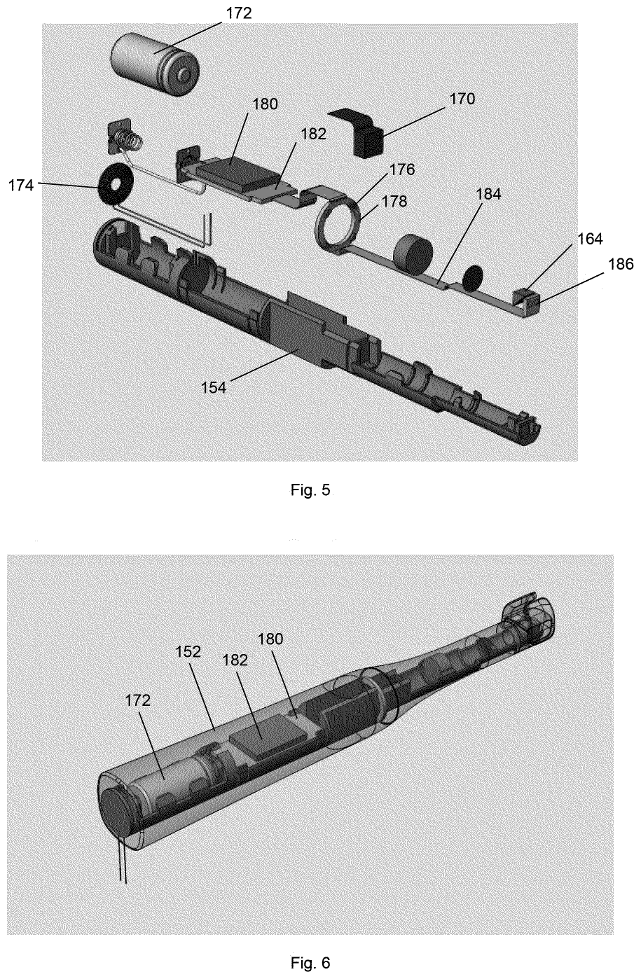

FIG. 5 shows an exploded view of components in the interior of the transillumination device;

FIG. 6 shows components in the interior of the transillumination device in their installed position;

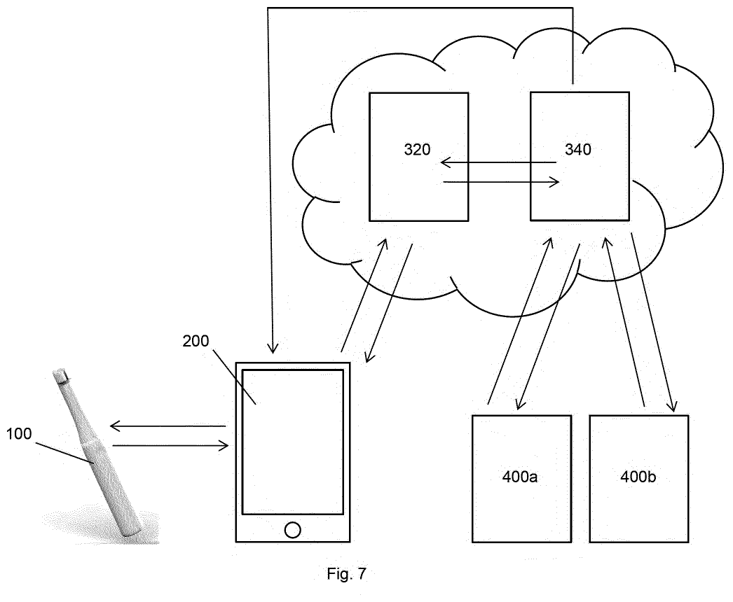

FIG. 7 shows a schematic of the system comprising a transillumination device, a user's smart device, and a remote server;

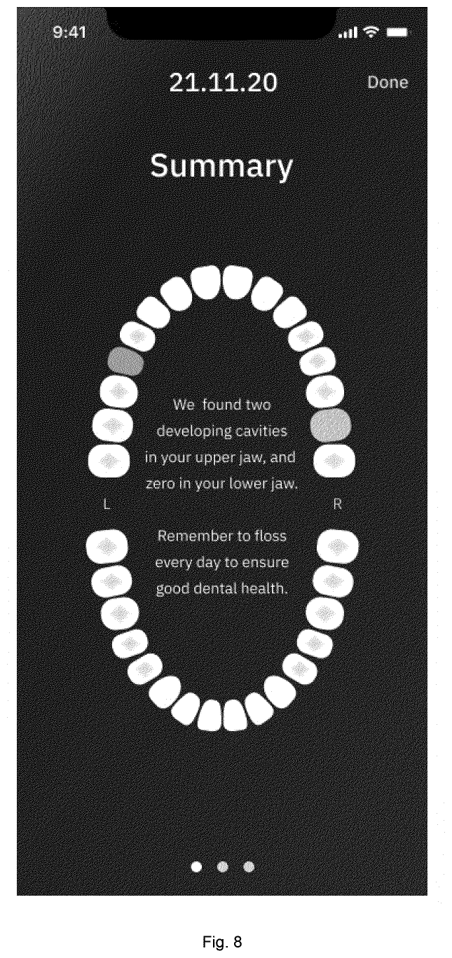

FIG. 8 shows an image of an app interface with the location of caries identified to the user.

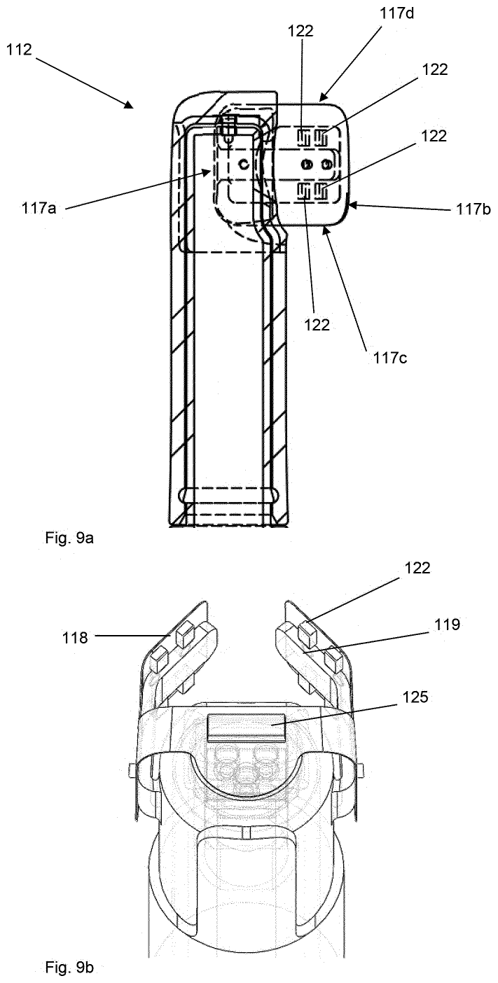

FIG. 9 shows the layout of light sources in the transillumination device;

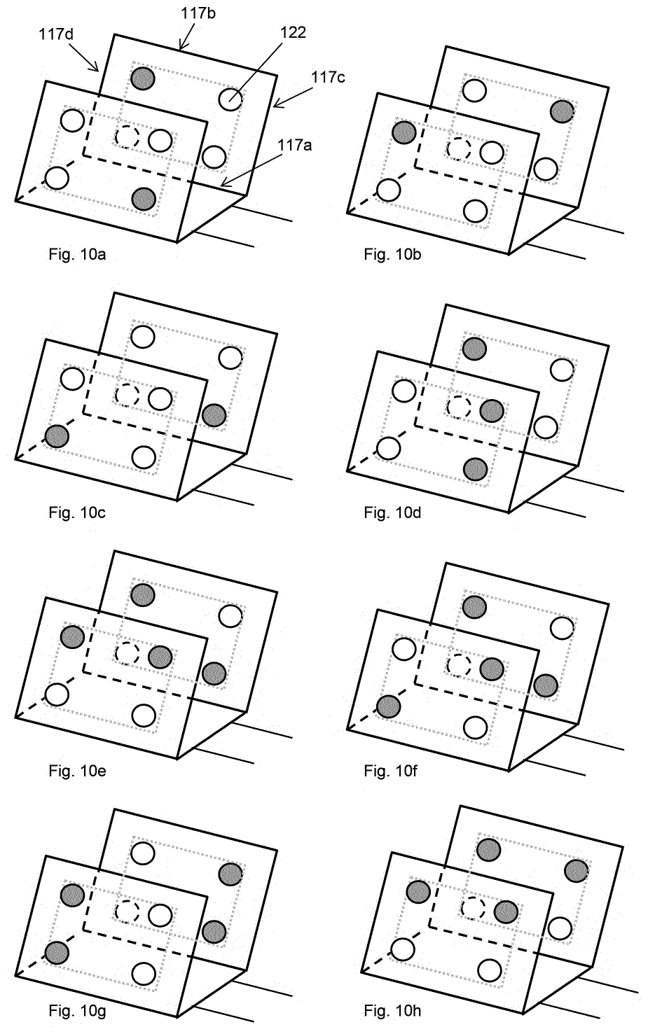

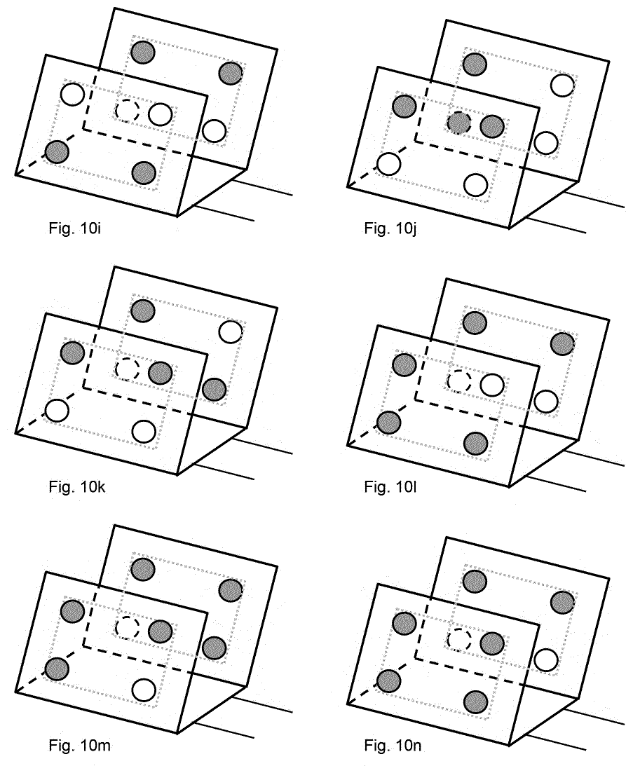

FIGS. 10a to 10n show exemplary lighting schemes demonstrating sets of light sources that can be illuminated or switched off, to give different lighting conditions;

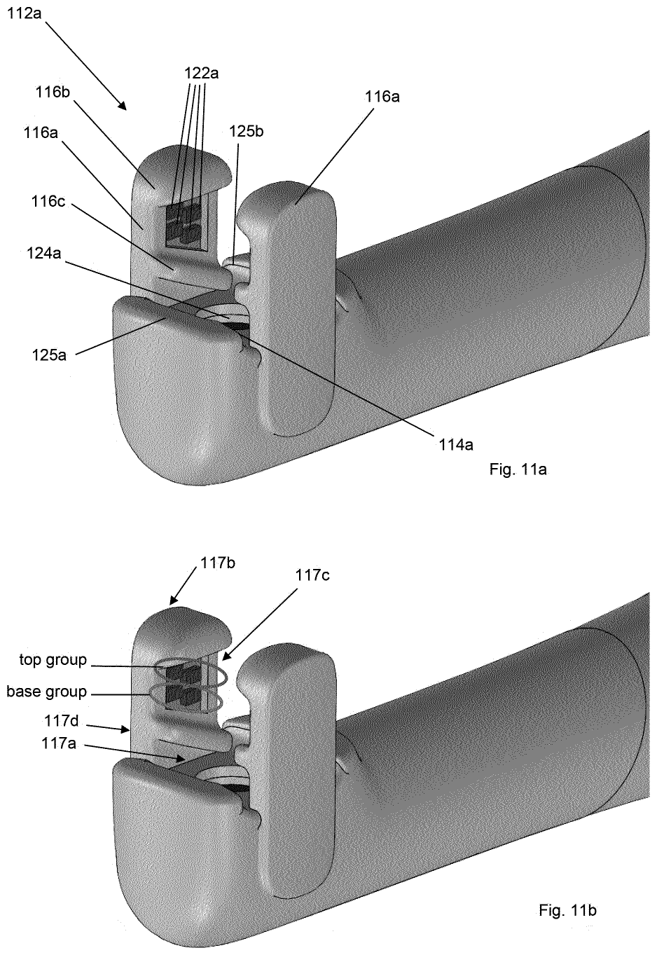

FIGS. 11a and 11b show an alternative configuration of the tip of the exemplary transillumination device;

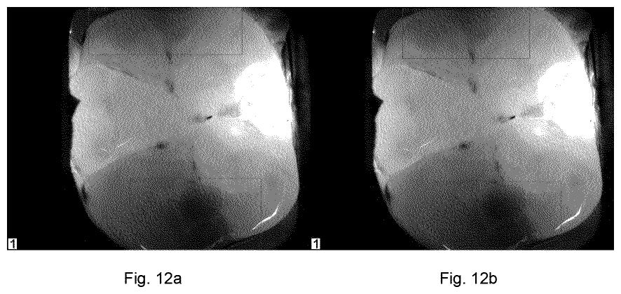

FIGS. 12a and 12b show images produced by transillumination with caries categorised as code 3;

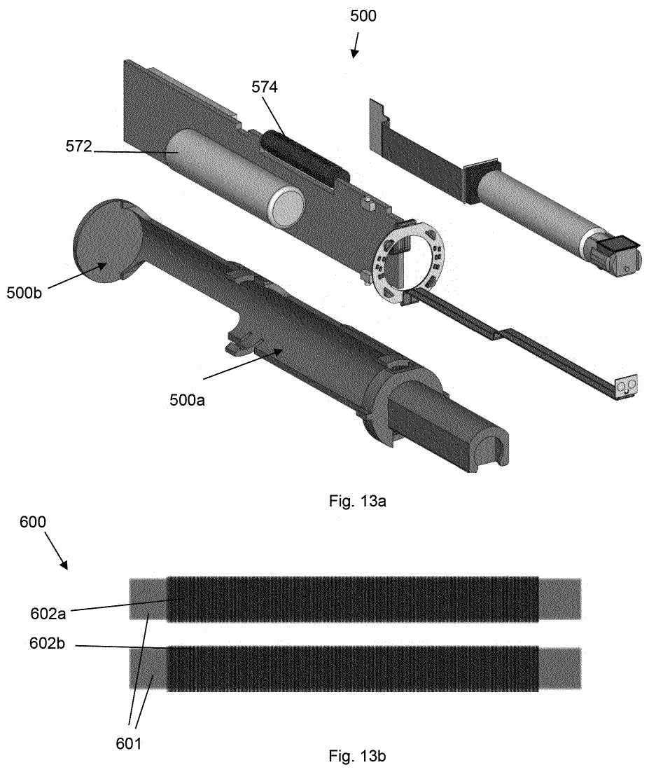

FIG. 13a shows an exploded view of components in the interior of a transillumination device, including an inductive charging coil for charging a rechargeable battery;

FIG. 13b shows a schematic view of an inductive charging arrangement;

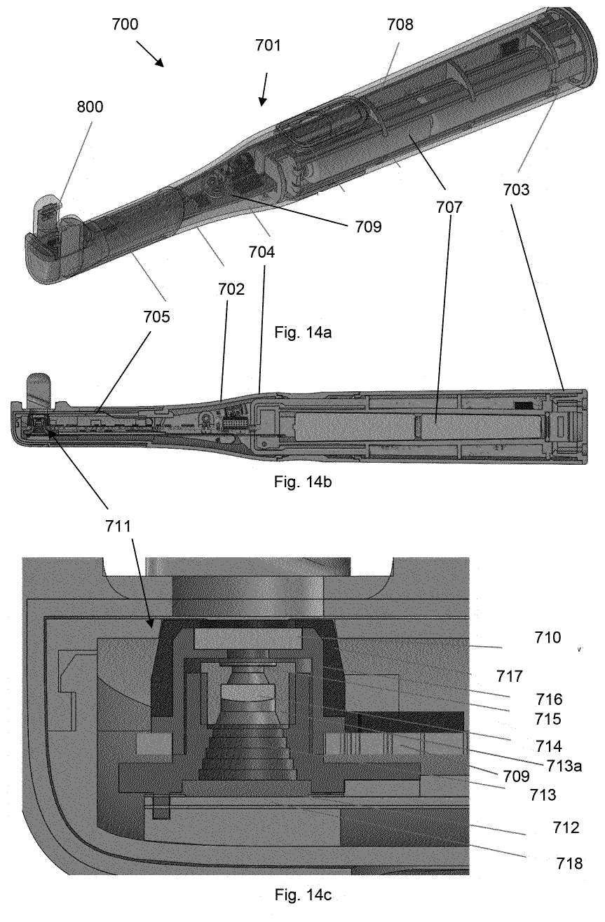

FIGS. 14a to 14c show an exemplary transillumination device comprising an optical assembly;

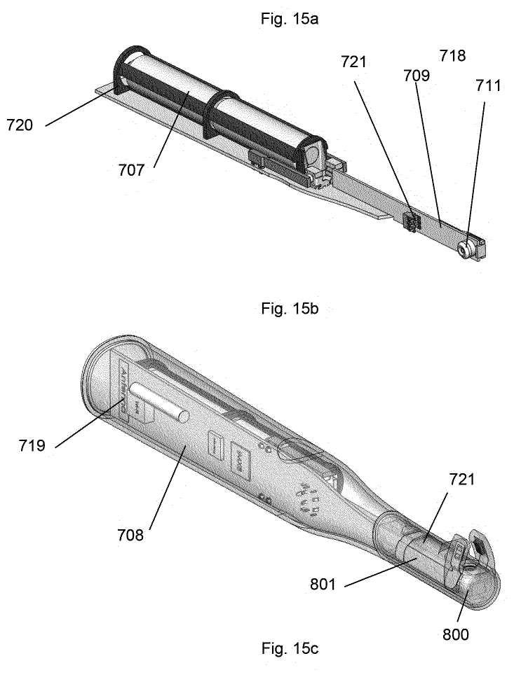

FIGS. 15a to 15c show the internal components of an exemplary transillumination device; and

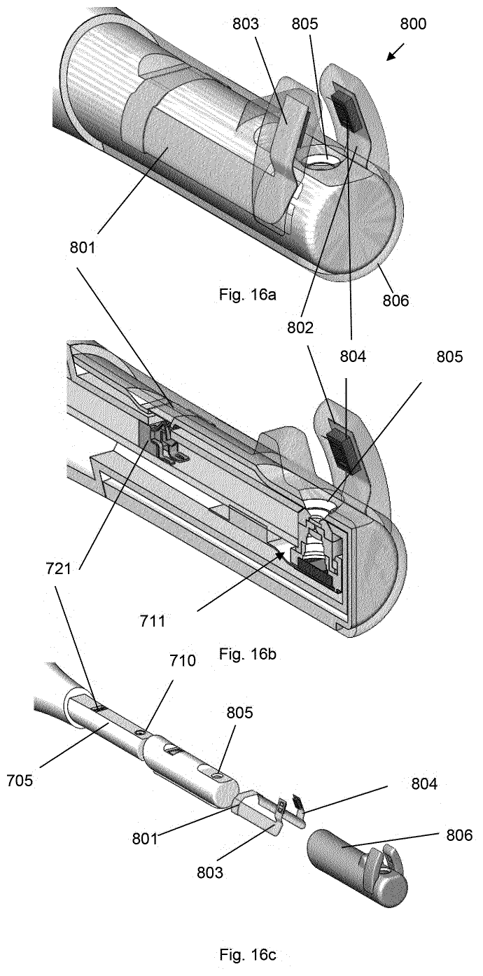

FIGS. 16a to 16c show an exemplary head portion of a transillumination device.

FIG. 1 shows an exemplary transillumination device 100. It will be appreciated that the device and method described herein could be used by a user on their own teeth (as described below) or by a first person on a second person's teeth.

The transillumination device comprises a head portion 110 and a main body portion 150. The head portion 110 comprises a tip 112 which is shaped so as to fit over a user's tooth.

FIG. 2 shows the tip 112 in more detail. The tip 112 comprises a central portion 114 with two flexible wing portions 116 extending therefrom. When the tip 112 is positioned on the user's tooth, the central portion 114 sits at or near the occlusal/incisal surface of the user's tooth, and the flexible wings contact either side of the tooth; one of the flexible wings sits against the buccal/facial side of the tooth, and the other sits against the lingual/palatal side of the tooth.

As shown in FIGS. 2 and 3, each of the flexible wing portions 116 comprises a support arm 119 which supports a flex PCB 118, both of which are over-moulded with a soft and flexible material 120 (in this case, a UV-curable elastomer is used but other materials may be used instead of the UV-curable elastomer; for example a thermoplastic elastomer (TPE) may be used). Each of the flexible wing portions 116 comprises four LEDs 122 attached to the flex PCB 118 and the four LEDs 122 are arranged in a rectangle configuration. By utilising a flex PCB 118, the flexible wings 116 can be made flexible to ensure that the LEDs 122 remain close to the tooth in use.

As discussed in more detail below, the transillumination device 100 is capable of applying a number of different lighting conditions to the tooth. This is achievable by having a plurality of LEDs in the tip 112.

Arranging the LEDs in a rectangular/square configuration or a parallelogram/rhombus configuration (or in any configuration where one or more LEDs are closer to the gum-line than one or more other LEDs, and where one or more LEDs are laterally offset from one or more other LEDs) on each side of the tooth ensures that one or more of the LEDs on each side is close enough to the tooth to transilluminate the tooth. Additionally, such an arrangement allows for the provision of light from several different angles (for example, close to the gum-line, and further up the tooth, and from two lateral directions) so that all evidence of caries within the tooth (seen as shadows on a transillumination image) can be imaged. Providing a plurality of LEDs with different positions provides a range of options for LED placement relative to the tooth, thus providing a technical solution to the problem of finding optimal LED placement for transillumination for a user at home (which normally requires extensive training for a dentist to realise in a clinic setting using a single light source). Providing a plurality of LEDs also allows for variable levels of illumination by turning on different numbers of LEDs. This allows to get an increased amount of light into a large tooth (for example, a molar), whereas a lesser amount of light is needed for transillumination of a small tooth (for example, an incisor).

Each LED 122 is configured to emit near-IR light. Here, near-IR light is defined as light having a wavelength in the range of 780 nm to 3000 nm (in accordance with the ISO 20473 standard). In this case, the LEDs have a peak wavelength at approximately 850 nm and a spectral bandwidth of approximately 35 nm. The radiant intensity (at a forward current of 100 mA) is approximately 9 to 18 mW/sr, and typically is around 13 mW/sr. The flexible wing portions 116 are configured to flex outwardly slightly when positioned over the tooth, but a returning force acts against this outward flexing, so that the tip 112 grips onto the sides of the tooth. The flexing of the flexible wing portions 116 ensures that there is minimal clearance between each LED 122 and the surface of the tooth; this reduces stray light which could be detrimental to imaging, and also ensures that an appropriate amount of light penetrates into the tooth to allow for imaging.

The LEDs 122 may also be configured to emit light in the visible spectrum—this allows a user to visualise that the device is working.

The head portion 110 also comprises a sleeve portion 126 comprising a hollow, broadly cylindrical portion. The sleeve portion 126 is configured to be received over a neck portion 160 of the main body portion 150, such that the head portion 110 and the main body portion 150 clip together.

The removable nature of the head portion 110 allows for several different users, e.g. members of the same family to use the transillumination device 100, with each family member using a different head portion 110. To allow for identification of each head portion 110 with a particular user, the head portion comprises an RFID tag (not shown). The RFID tag is read by a user's smart device (e.g. a mobile telephone 200 having a display screen, as shown in FIG. 7). Alternatively near-field communication (NFC) between the transillumination device 100 or head portion 110 and the user's smart device could be used to identify each head portion 110.

The central portion 114 of the tip 112 comprises a window 124 in the over-moulding 120. The window 124 is configured to face the occlusal/incisal surface of the user's tooth. When the head portion 110 is clipped onto the main body portion 150, the window 124 in the central portion 114 of the tip 112 aligns with a corresponding window 162 in the neck portion 160 of the main body portion 150.

In use, light from the tooth passes through the window 124 in the central portion 114, through the corresponding window 162 in the neck portion 160, and into the neck portion 160. The neck portion 160 holds an optical assembly for directing the light to a camera 170. The optical assembly (shown in FIG. 4) comprises a prism 164 which receives light from the window 162 and bends it to direct it along the direction of the axis of the neck portion 160. Further optical components (an aperture 166 and three plano-convex lenses 168) in the optical assembly shape the light beam and focus it onto the imaging area of the camera 170. It will be appreciated that the optical arrangement may be adapted for use with different wavelengths of light from the LEDS.

In this case, the camera 170 is a low-voltage CMOS device. The camera outputs colour images, which are converted to greyscale for further analysis. The transillumination device 100 may carry out such a conversion or this can be performed by user's smart device (in this case a mobile telephone 200). As an alternative, a monochromatic camera could be used. There may be filters, for example a software filter, coupled to the camera to filter out different parts of the spectrum. Image processing of captured images may be performed by the transillumination device 100 and/or the user's smart device 200 and/or by a remote server 300. Such image processing may include filtering by wavelength, exposure control, white balance, colour saturation, hue control, white pixel cancelling, and/or noise cancelling.

FIG. 5 shows other internal components in the main body portion 150 of the transillumination device 100 in an exploded view. These components are shown in their installed position in FIG. 6. The transillumination device 100 is battery powered, and comprises a rechargeable battery 172. The battery 172 is inductively charged by an inductor 174 which interacts with an inductive charging station, of the kind known in the art. Similar inductive charging stations are for example commonly used to charge electrical toothbrushes. Alternative power and charging systems are shown in FIGS. 13a and 13b, discussed in more detail below.

The transillumination device 100 comprises means for providing feedback to the user. This takes the form of a vibration motor (not shown) and a plurality of user-feedback LEDs 176 (in this case four) radially spaced around an annular portion 178 of a flex PCB 184, and which emit visible light through the material forming main body portion 150. Each LED 176 is an RGB LED (i.e. comprising a combination of 3 LEDs: one red; one green; and one blue in just a single package). In this way many colors of light can be produced by each LED. The feedback comprises an indication to the user that suitable images were or were not captured by the device in use (as discussed in more detail below). Alternatively or additionally, similar feedback can be provided by the user's smart device.

Control of the operation of the transillumination device 100 is by a controller 180 in the form of a system-on-a-chip microcontroller. The controller 180 comprises integrated Wi-Fi/Bluetooth capability. The controller may perform any of the image processing mentioned above. The controller 180 is located on a main PCB 182 which is connected to the battery 172. The main PCB 182 is also connected to the flex PCB 184. The flex PCB 184 comprises the annular portion 178 and an LED-interface portion 186 located at the end of the neck portion 160, as well as connector portions of the flex PCB 184 which run between the main PCB to the annular portion 178 and from the annular portion 178 to the LED-interface portion 186.

All connections between the controller 180 and the LEDs 176 on the annular portion 178 run along the flex PCB 184 (and onto the main PCB 182), and similarly all connections between the controller 180 and the connectors on the LED-interface portion 186 run along the flex PCB 184 (and onto the main PCB 182).

Forming the annular portion 178 and LED-interface portion 186 as portions of a flex PCB reduces the cost and complexity of assembly; if the annular portion 178 and LED-interface portion 186 were instead formed from rigid PCBs, then additional cable connections to the main PCB 182 would be needed. Moreover, using a flex PCB 184 enables the device to be kept compact; a flex PCB is very flat which enables a thinner neck portion 160 to be provided. Since cable connectors are not needed, this also allows to keep the size down.

A flex PCB is also used because its flexibility can be utilised to bend the PCB into a suitable shape—so the annular portion 178 and LED-interface portion 186 are arranged in a transverse direction to the axial extent of the main body portion 150.

When the head portion 110 is clipped onto the main body portion 150, the LED-interface portion 186 is electrically connected to the flex PCB 118 in the head portion 110. This connection is achieved via three pogo connectors 127 (see FIG. 3b) which are connected to the flex PCB 118 in the head portion 110. These pogo connectors 127 pass through small holes 128 (see FIG. 3a) in the top of the sleeve portion 126 and through corresponding small holes 163 in the top of the neck portion 126, and contact the LED-interface portion 186 of the flex PCB 184. Whilst two small holes 128 and two corresponding small holes 163 are shown in FIG. 3a, three of each would be provided—one for each of the three pogo connectors 127.

The flex PCB 118 comprises an LED controller chip 125 for controlling each of the LEDs 122 individually (i.e. each LED 122 can be separately controlled independently of the status of any of the other LEDs 122). The control of the LEDs 122 is done automatically by the device, without any input from the user.

The main body portion 150 comprises an outer housing 152 (shown in FIG. 6) and an inner cradle 154. The inner cradle 154 comprises a series of resilient projections designed to form areas into which the various components can be clipped. Therefore, the inductor 174, battery 172, main PCB 182, flex PCB 184 (including the annular portion 178 and LED-interface portion 186), and camera 170 are all secured within the inner cradle 154 without use of any screws. This facilitates ease of assembly/maintenance and reduces manufacturing costs.

The transillumination device 100 may be turned on/off using a user's smart device 200. This control (and any other communication between the smart device 200 and transillumination device 100) can occur via a Bluetooth connection. Alternatively, one or more buttons may be present on the main body portion 150 for this purpose.

The prism 164, aperture 166 and three plano-convex lenses 168 are held within a portion of the inner cradle 154 extending into the neck portion 160. The lenses are held in cylindrical spacers to ensure that the correct optical path is maintained. Again, no screws are needed for securing the components of the optical assembly.

The outer housing 152 is formed from a durable and waterproof plastic, such as acrylonitrile butadiene styrene (ABS) plastic.

FIG. 7 shows a system for enabling detection of caries by a user at home, of which the transillumination device 100 is a part. Also part of the system are a user's smart device (in this case a mobile telephone 200 having a display screen but it will be appreciated that any suitable smart device could be used including a tablet, a laptop, desktop or a purpose-built smart device) and a remote server, e.g. a cloud-based server 300. As noted above, the transillumination device 100 comprises a system-on-a-chip microcontroller with integrated Wi-Fi/Bluetooth capability. This allows communication between the transillumination device 100 and the mobile telephone 200. The mobile telephone 200 comprises a communications interface (as is known in the art) capable of communicating with the cloud-based server 300 via the internet.

In brief, the mobile telephone 200 runs a software application (an app) which receives images from the transillumination device 100, performs processing based on those images, and then transmits the images to the cloud-based server 300. The cloud-based server 300 performs any further necessary processing of the images which has not already been carried out by the mobile telephone, and sends the results back to the mobile telephone 200, to be displayed to the user on the display screen of the mobile telephone 200.

Before using the transillumination device 100 for the first time, the user opens the app and is taken through a process whereby they register.

Registration of the user comprises the setting of a username and password, registration of an email address, acceptance of terms and conditions, selection of a subscription package if applicable, entering of payment details, and connection to the user's smart device. These registration steps can later be amended.

Registration may be performed for other members of the same household to use the same device.

The user also registers with the app if the user has any missing teeth, fillings in any of their teeth, implants, prosthetics or any other know conditions. Information about the user's diet and/or health conditions may optionally also be collected.

The image capturing process is now described in greater detail. The app running on the mobile telephone 200 guides the user during the image capturing process. The user is directed to move the transillumination device 100 around one quadrant (i.e. quarter) of the jaw at a time, as described below. Each quadrant is defined between the rearmost tooth to the central front incisor, for the upper and lower jaw and for the left and right sides of each—i.e. there is an upper-left quadrant, a lower-left quadrant, an upper-right quadrant and a lower-right quadrant.

For each quadrant, the user is instructed where to start (e.g. from the rearmost tooth of one of the four quadrants) and accordingly places the head portion 110 of the transillumination device 100 over the rearmost tooth (or other designated starting point) of the selected quadrant and slides it into place so that the central portion 114 is facing the occlusal/incisal surface of the tooth, with the flexible wing portions 116 contacting either side of the tooth. Then the user turns on the image capturing, for example by clicking a button on the device or the app and, under the direction of the app, moves the head portion 110 from tooth to tooth until all teeth in the selected quadrant are imaged. Under the direction of the app, the user briefly pauses (e.g. for 1 to 3 seconds) over each tooth to be imaged so that the head portion 110 is temporarily held stationary relative to the tooth being imaged. Whilst held stationary, different lighting conditions are applied to the tooth (as discussed below), and a plurality of imaged are captured. Each image is sent to the mobile telephone 200 for processing by the app.

Alternatively, the transillumination device may perform a pre-selection of the images, based on image quality, so that only images meeting certain quality criteria are sent to the mobile telephone 200. The quality criteria include one or more of focus/sharpness, acceptable lighting (i.e. not over/under exposed) and the presence of a tooth within the image.

The app comprises an algorithm that detects if a tooth is present within an image. In this algorithm the tooth image is segmented, and shape recognition is used to determine the type of tooth. Together with the given quadrant, and the known sequence of the teeth, the imaged tooth is identified and labelled according to the international standard numbering system. Based on the position of the tooth, and the brightness and the contrast in the image, the app selects the best image for each tooth from the series of images that are captured for that tooth. The selection may be based on quality of the images such as uniformity of lighting, contrast and alignment of the image. When/if the quality of the image is approved (i.e. it meets predetermined criteria such as those outlined above), visual feedback is provided to the user via their smart device to indicate which teeth have successfully been imaged. For example, the corresponding tooth will be marked on the model of the teeth presented on the app (for example, the corresponding tooth may be shaded green), and the user is instructed to move to the next tooth. If no images are approved for one or more teeth because of insufficient quality for analysis, the app indicates to the user that the tooth should be re-imaged.

As an alternative to the user holding the head portion 110 stationary on each tooth to acquire images, the user may sweep the head portion 110 across the quadrant with a slow, continuous sweeping motion. A series of images are taken in the sweep, including images under different lighting conditions for each tooth. If one or more of the teeth is not successfully imaged, the app directs the user to perform the sweep again.

Once an image of each tooth is approved and labelled, the images are then sent from the mobile telephone 200 to the cloud-based server 300. Such images can be anonymised prior to communication and the communication can be end-to-end encrypted to protect a user's privacy. The cloud-based server 300 comprises a machine learning algorithm, which in this case is a deep learning neural network (running on a processor 320) with the capability of identifying caries and categorising the identified caries in transillumination images of the teeth.

The processor 320 returns a user report regarding the status of each tooth (i.e. no caries, or caries, and the classification of caries if they are present) to the mobile telephone 200 and this is displayed to the user as visual feedback on a picture of the arrangement of the teeth (as shown in FIG. 8), showing the mapping of teeth and their status.

The user report generated by the processor 320 is encrypted prior to sending to the mobile telephone 200. The report does not include any information which would allow the user to be identified, i.e. the data is anonymised.

The app displays informative and/or motivational text (for example, “Well done!”) if no caries are identified. If caries code 1 or 2 are identified, helpful information regarding appropriate oral care (for example, “Ensure that you brush for at least two minutes in the morning and the evening”) is displayed to the user.

If any of the caries are identified as code 3, 4 or 5, the user is advised to visit a dentist. The cloud-based server 300 comprises a database of dental clinics, and the app presents a list of such clinics which are local to the user (and/or a map showing the location of these), based on the user's location data or the user selecting a locality. Alternatively such a database may be included in the app or it may be a separate database in its entirety, which can be accessed by the cloud-based server or smart device. The user can choose one or more dental clinics 400a, 400b from this list to be given access to a detailed dental report, so that the dental clinic(s) can provide a treatment plan and/or cost estimate to the user for treatment of the identified caries. The detailed dental report is a standardised report including images of all of the user's teeth captured by the transillumination device 100. The detailed dental report comprises a clickable model of the teeth so that the dental professional can select each individual tooth to see the corresponding transillumination image and labelled caries. The detailed dental report does not include any information which would allow the user to be identified, i.e. the data is anonymised.

The user is also given the option to add an email address for a dental clinic (not already in the database) that they would like to send the report to. A link to download the report is sent to that email address, and the dental clinic would then have to sign up and log in to access to the report.

To facilitate the dental clinics accessing the user's report, as well as sending the user's report to the mobile telephone 200, the processor 320 sends the detailed dental report to a safe server 340. The server is safe in that all communication with the safe server 340 is end-to-end encrypted, and two-step authentication is required to access data from the safe server 340.

When a user chooses one or more dental clinics to be given access to their report, a link is sent from the safe sever 340 to the dental clinic(s) 400a, 400b to enable them to access the report from the safe server 340.

As noted above, communication between the dental clinic(s) 400a, 400b and safe server 340 is end-to-end encrypted, and the dental clinic(s) 400a, 400b are authenticated by two-factor authentication. Once authenticated, the dental clinic is able to download the user's report from the safe server 340.

A dental practitioner at the dental clinic reviews the images and the identified caries, and determines a treatment plan and/or cost estimate and may suggest an appointment time/location. This information is then sent back to the safe server 340. The safe server 340 receives the cost estimate and/or treatment plan and delivers this via the app to the user. The user can then contact a dental clinic 400a, 400b to arrange treatment.

The lighting features of the transillumination device 100 will now be described in greater detail. As noted above, the first and second flexible wing portions 116 each comprise a plurality of LEDs. The LEDs 122 are individually controllable, and each is connected to the controller 180 via the flex PCB 184 in the main body portion 150 (comprising the LED-interface portion 186), the pogo connectors, and the flex PCB 118 in the head portion 110. Because the LEDs 122 are individually controllable, the transillumination device 100 has the capability of illuminating teeth with different sets of LEDs illuminated, so as to create a plurality of different lighting conditions.

FIGS. 9 and 10 give further details regarding the positioning and control of the LEDs 122 in the tip 112.

Each flexible wing portion 116 is defined by four edges. These are labelled in FIG. 9. Firstly, there is a base edge 117a, where the flexible wing portion 116 meets the central portion 114, and an opposed opening edge 117b, where the flexible wing portion defines the slot for receiving a tooth. Then, transverse to the base edge and opening edge are the proximal edge 117c (nearest to the main body portion 150 of the device) and the opposed distal edge 117d (furthest from the main body portion 150 of the device).

Each flexible wing portion 116 comprises four LEDs 122. As shown in FIG. 9, these are arranged so as to be positioned at the corners of a rectangle. Here, two sides of the rectangle are broadly parallel to the base edge 117a and the opening edge 117b, and two sides of the rectangle are broadly parallel to the proximal edge 117c and the distal edge 117d. The LEDs 122 are spaced apart by 1.75 mm (the perpendicular distance from edge-to-edge) along the sides of the rectangle parallel to the proximal edge 117c and the distal edge 117d, and are spaced apart by 5 mm (the perpendicular distance from edge-to-edge) along the sides of the rectangle parallel to the base edge 117a and the opening edge 117b.

The LEDs are individually controllable (such that each can be turned on or off independently of the status of the others), and this allows for multiple different lighting conditions to be achieved.

FIGS. 10a-10n show schematically different lighting conditions that can be obtained. The arrangements show are not exhaustive or limiting in any way, but are simply illustrative of some of the many lighting conditions which could be achieved. In these figures, an illuminated LED is represented as a shaded circle. In these figures, the distances between the LEDs have been exaggerated for ease of illustration.

In general, at least one LED is illuminated on each side. This is advantageous, but not limiting on the invention. Similarly, it is generally advantageous light is illuminated further from the tip of the tooth (i.e. closer to the gum-line). The camera captures images from near the tip of the tooth, so to image the greatest possible volume of the tooth, and in particular to capture as much as possible of the shadows indicative of caries, it is advantageous to have light entering the tooth further from the tip of the tooth, near the gum-line. If the tooth is illuminated too far from the gum-line, the shadows nearer the gum-line will be hidden.

FIGS. 10a-10c show lighting conditions with two LEDs illuminating the tooth at the same time. FIGS. 10d and 10e show lighting conditions with three LEDs illuminating the tooth at the same time. FIGS. 10f-10k show lighting conditions with four LEDs illuminating the tooth at the same time. FIG. 101 shows a lighting condition with five LEDs illuminating the tooth at the same time, and FIGS. 10m-10n show lighting conditions with six LEDs illuminating the tooth at the same time. Seven, or even all eight of the LEDs could also be lit at the same time.

As discussed above, the LEDs can each be controlled such that each is either on, or off. However, instead of an LED being off, it could instead be illuminated at a reduced intensity.

Whilst eight LEDs 112 have been described above, a greater number of (or fewer) LEDs could instead be provided. Additional lighting conditions would be available for a greater number of LEDs.

Whilst the LEDs described above all have the same nominal spectral characteristics (including the same peak wavelength and peak width), it will be appreciated that one or more of the LEDs could have different spectral characteristics from the others (with peak wavelengths still within the near-IR range) to allow for images to be captured under a plurality of near-IR wavelengths.

The plurality of lighting conditions can be cycled through as the transillumination device 100 is moving over the teeth, so that for each tooth, at least one image is taken under each of the plurality of lighting conditions. At least one of the plurality of lighting conditions should result in the capture of a suitable image for caries analysis, but the particular lighting condition which will achieve this will vary depending on the tooth being imaged. For example, for a molar which has had a filling, a suitable lighting condition could be that all the LEDs 122 should be illuminated. However, when an incisor is imaged under the same lighting condition, it is likely that the resulting image would be over-exposed (because too much light was incident into the tooth). In that case, a suitable lighting condition would include only a subset of the LEDS 122 (i.e. not all of the LEDs 122) begin illuminated. For example, half of the LEDs could be illuminated.

As an alternative to cycling through all lighting conditions for each tooth, the lighting conditions may be applied based on knowledge of the particular tooth that is being imaged, with a predetermined lighting condition being applied for that particular tooth. Knowledge of the particular tooth that is being imaged may be obtained through shape-recognition of the tooth (for example using machine learning techniques and based on geometrical patterns), and/or through knowing the expected sequence of teeth to be imaged, for example. If the resulting image is not acceptable (because the lighting condition does not allow the tooth to be imaged correctly), then a different lighting condition may be applied.

The first lighting condition to be applied to a given tooth may be pre-set based on a prior calibration process carried out by the user on first use of the device. In the calibration process, a plurality of lighting conditions may be sequentially applied to the tooth, and an image may be acquired under each lighting condition. Then, the images are analysed to determine the highest quality image. The lighting condition under which the highest quality image was acquired is then pre-set as the first lighting condition. The highest quality image may for example be one with minimal reflections from stray light, and/or an image with the correct exposure (not overexposed by putting too much light into the tooth, or underexposed by putting too little light into the tooth), and/or an image with appropriate brightness and contrast.

It will be appreciated that rather than using a single captured image, it is possible to capture and select multiple images of the same tooth and use parts of each image, or multiple overlaid images of the same tooth in the trained neural network of the remote server.

For example, it is possible to capture multiple images of a tooth under illumination by near-IR at different wavelengths (for example where one or more LEDs are provided having different wavelength characteristics from the others). The multiple images of the same tooth can then be combined to make a combined image as an input to the machine learning algorithm. Each image of the multiple images in the combined image can be combined with the others with a weighting factor of between 0 and 1, where 0 means no contribution and 1 means complete contribution.

FIGS. 11a and 11b show the tip 112a of a head portion of a transillumination device, similar to the tip shown in FIG. 2. The tip 112a comprises a central portion 114a with two flexible wing portions 116a extending from the central portion 114a. When the tip 112a is positioned on the user's tooth, the central portion 114a sits at or near the occlusal/incisal surface of the user's tooth, and the flexible wing portions contact either side of the tooth; one of the flexible wing portions sits against the buccal/facial side of the tooth, and the other sits against the lingual/palatal side of the tooth. Each of the flexible wing portions 116a comprises four LEDs 122a. These are attached to a flex PCB (not shown) supported by a support arm (not shown) forming part of each of the flexible wing portions 116a.

The flexible wing portions 116a and central portion 114 comprise a soft and flexible over-moulding. In this case, a UV-curable elastomer is used for the majority of the over-moulding, with an IR-transparent silicone material being used for the over-moulding over the LEDs 122a. Other materials may be used instead of the UV-curable elastomer; for example a thermoplastic elastomer (TPE) may be used. The central portion 114a of the tip 112a comprises a window (i.e. an aperture) 124a in the over-moulding. The window 124a is configured to face the occlusal/incisal surface of the user's tooth.

The flexible wing portions 116a are flexible to ensure that the LEDs 122a remain close to the tooth in use. The flexible wing portions 116a are configured to flex outwardly slightly when positioned over the tooth, but a returning force acts against this outward flexing, so that the flexible wings 116a grip onto the sides of the tooth. The flexing of the flexible wing portions 116a ensures that there is minimal clearance between each LED 122a and the surface of the tooth; this reduces stray light which could be detrimental to imaging, and also ensures that an appropriate amount of light penetrates into the tooth to allow for imaging.

Each LED 122a is configured to emit near-IR light, having a peak wavelength at approximately 850 nm and a spectral bandwidth of approximately 35 nm. The radiant intensity (at a forward current of 100 mA) is approximately 9 to 18 mW/sr, and typically is around 13 mW/sr. The LEDs 122a may also be configured to emit light in the visible spectrum—this allows a user to visualise that the device is working.

Each flexible wing portion 116a is defined by four edges. These are labelled in FIG. 11b. Firstly, there is a base edge 117a, where the flexible wing portion 116a meets the central portion 114a, and an opposed opening edge 117b, where the flexible wing portion defines the slot for receiving a tooth. Then, transverse to the base edge and opening edge are the proximal edge 117c (nearest to the main body portion 150 of the device) and the opposed distal edge 117d (furthest from the main body portion 150 of the device).

As shown in FIG. 11a, two LEDs are located so as to be further from the tip of the tooth when the head portion is in place on a tooth (the upper two LEDs as illustrated in FIG. 11a, closest to the base edge 117a), and two LEDs are located so as to be positioned closer to the tip of the tooth when the head portion is in place on a tooth (the lower two LEDs as illustrated in FIG. 11a, furthest from base edge 117a). The two LEDs located so as to be further from the tip of the tooth when the head portion is in place on a tooth are vertically offset from one another. The two LEDs located so as to be closer to the tip of the tooth when the head portion is in place on a tooth are vertically offset from one another. The vertically offset position allows to cover a slightly larger extent of the tooth in the vertical direction, compared to the configurations shown in previous figures, with side-by-side LEDs.

That is, the four LEDs 122a on a flexible wing portion 116a sit at the corners of a rhombus/parallelogram. Two sides of the rhombus/parallelogram are parallel to the proximal edge and the distal edge 117d, and the other two sides are non-parallel to the base edge 117a.

At least one LED 122a should be located adjacent to the gum-line when in use, no matter which tooth is being imaged. Here, adjacent to the gum-line means close to, but slightly vertically offset from the gum-line, so that light is still directed into the tooth rather than into the gum. To image the greatest possible volume of the tooth, and in particular to capture as much as possible of the shadows indicative of caries, it is advantageous to have light entering the tooth from near the gum-line. It depends on the tooth as to which of the LEDs 122a is in the best position; on a molar in the lower jaw for example the best-positioned LED 122a may be the one positioned lowermost down the tooth when the head portion is in place on a tooth. On an incisor or canine in the lower jaw, for example, that same LED 122a may be located below the gum-line, in which case it is not best-positioned to illuminate the tooth. In such a case, the best positioned LED 122a may be one located closest to the tip of the tooth when the head portion is in place on the tooth.

The flexible wing portions 116a shown in FIGS. 11a and 11b each comprise two projections facing towards the opposite flexible wing portion 116a (i.e. facing inwardly towards the slot between the flexible wing portions 116a which receives the tooth in use). A first projection 116b on a flexible wing portion 116a runs along the opening edge 117b of the flexible wing portion 116a. This first projection 116b is in order to position the LEDs at a small distance from the surface of the tooth. This allows for the LED light to be more evenly spread, compared to the case where the LEDs were closer to the tooth. When the LEDs are too close to the tooth, there may be an uneven spread of light, and a risk of partial overexposure in the captured images.

The second projection 116c runs from the proximal edge 117c (nearest to the main body portion 150 of the device) to the opposed distal edge 117d (furthest from the main body portion 150 of the device), and is located between the LEDs 122a and the base edge 117a. This second projection 116c should prevent or reduce light from being transmitted directly from the LEDs 122a into the camera. In the embodiment shown, the second projection 116c slopes down towards the base edge 117a in the direction from the distal edge 117d to the proximal edge 117c.

The central portion 114a comprises alignment projections 125a and 125b. The first alignment projection 125a projects away from the central portion 114 and runs across the top of the tip 112a (where the top of the tip is the end of the tip furthest from the main body portion 150). The second alignment projection 125b is broadly parallel to the first alignment projection 125a, but is located the other side of the flexible wing portions 116a than the first alignment projection 125a. The first and second alignment projections therefore bracket the flexible wing portions 116a. The first and second alignment projections are broadly perpendicular to the flexible wing portions 116a. The first and second alignment projections aid the user to hold the device parallel to the row of teeth.

As shown in FIG. 11b, in this embodiment, on each flexible wing portion 116a there are two groups of two LEDs—a base group (the two LEDs closest to the base edge 117a) and a top group (the two LEDs furthest from the base edge 117a). Each group is independently controllable, so there are a total of four independently controllable groups. Labelling one flexible wing portion 116a as the “left-hand” wing, and one as the “right-hand” wing, we have:
a right-hand base group;
a right-hand top group;
a left-hand base group; and
a left-hand top group.

Some exemplary lighting conditions (combinations 1 to 10) which can be achieved with such a configuration are listed below. Here, only the groups which are illuminated are mentioned.

The other groups which are not mentioned are not illuminated.
Combination 1—both base groups (left-hand and right-hand) lit
Combination 2—both top groups (left-hand and right-hand) lit
Combination 3—right-hand base group lit
Combination 4—right-hand top group lit
Commination 5—left-hand base group lit
Combination 6—left-hand top group lit
Combination 7—right-hand top and right-hand base groups lit
Combination 8—left-hand top and left-hand base groups lit
Combination 9—right-hand base group and left-hand top group lit
Combination 10—right-hand top group and left-hand base group lit

In these combinations, the maximum number of LEDs lit simultaneously is four, but more may be used. Using four or fewer LEDs at any one time reduces the risk of reflection of the LED light, which can be detrimental to image quality.

For a given tooth, a pre-set combination (or combinations) is used. These may be identified in a calibration process carried out by the user where on first usage of the device, all of the combinations will be cycled through for each tooth, and an appropriate condition/conditions will be identified for each tooth. The appropriate combination(s) depends on tooth size (height and thickness) and position.

Combinations 3 to 6 provide light coming from only one side of the teeth. These combinations are particularly suitable for imaging incisors. The light should then be directed from the labial (facial) side of the incisor, through the tooth, and out of the lingual/palatal side of the tooth. This is because the lingual/palatal side of an incisor has a sloping shape, which can create strong reflections of the LED light, leading to poor imaging. The incisors are thinner than other teeth, and images of sufficient quality can be obtained with light coming from only one side. Whether a base group of top group is used for a given tooth depends on the height of the tooth.

Combinations 7 to 9 might be used for example when imaging very large molars, where additional light is needed to obtain images of sufficient quality.

The groups of LEDs can each be controlled such that every LED in a group is either on, or off. However, instead of a group being off, the LEDs within that group could instead be illuminated at a reduced intensity.

Whilst eight LEDs 112a have been described above, a greater number of (or fewer) LEDs could instead be provided.

Whilst the LEDs 122a described above all have the same nominal spectral characteristics (including the same peak wavelength and peak width), it will be appreciated that one or more of the LEDs could have different spectral characteristics from the others (with peak wavelengths still within the near-IR range) to allow for images to be captured under a plurality of near-IR wavelengths.

Rather than using a single captured image, it is possible to capture and select multiple images of the same tooth and use parts of each image, or multiple overlaid images of the same tooth in the trained neural network of the remote server.

For example, it is possible to capture multiple images of a tooth under illumination by near-IR at different wavelengths (for example where one or more LEDs are provided having different wavelength characteristics from the others). The multiple images of the same tooth can then be combined to make a combined image as an input to the machine learning algorithm. Each image of the multiple images in the combined image can be combined with the others with a weighting factor of between 0 and 1, where 0 means no contribution and 1 means complete contribution.

The control scheme identified above (with four groups of two LEDs, operable in some or all of the combinations listed above) is not limited to usage with the tip 112a shown in FIGS. 11a and 11b, but could instead be implemented with the tip 112 discussed in relation to the earlier Figures.

FIGS. 12a and 12b show images produced by transillumination with a caries categorised as code 3. The image on the left shows the caries identified by a dentist as category 3 caries, whereas the image on the right shows the regions where the machine learning model identified the location of caries, which were also categorized as code 3 by the machine learning model.

The machine learning algorithm is trained on a training set of images comprising 1000 images of each category of caries (category 1 to category 5), as identified by a qualified dentist.

Whilst one configuration for a transillumination device has been described above, other variations are possible. Some possible alternative configurations are described below.

As discussed above, one configuration of the transillumination device comprises a rechargeable battery 172 which is inductively charged by an inductor 174. The inductor 174 sits in the base of the main body portion, and interacts with an external inductive charging station, of the kind known in the art. Similar inductive charging stations are for example commonly used to charge electrical toothbrushes. An alternative configuration is discussed below.

It is advantageous for the transillumination device to be able to communicate wirelessly (via Bluetooth for example) with the user's smart device (e.g. mobile phone) whilst the transillumination device is in use, i.e. being held by the user in their hand. Since water in the user's body (e.g. in their hand/mouth) absorbs Wi-Fi signals (e.g. 2.45 GHz radio waves), it is advantageous to position the Wi-Fi antenna at a position at which the user's hand or mouth does not surround the Wi-Fi antenna. A suitable position is the base of the main body portion of the transillumination device. However, the inductor (comprising ferrite material) forming part of the indicative charging circuit also absorbs Wi-Fi signals (e.g. 2.45 GHz radio waves). For this reason, to allow the Wi-Fi antenna to be positioned in the base, the indicative charging circuit must be moved away from the conventional position in the base.

FIG. 13a shows an exploded view of components in the interior of a transillumination device 500. In the configuration shown, the inductor 574 for charging the rechargeable battery 572 is located in a middle portion 500a of the transillumination device 500. There is also a Wi-Fi antenna (not shown) positioned in the base portion 500b of the device. Thus, the Wi-Fi antenna is in a position where it is unobstructed by the user or the inductor 574.

It will be appreciated that with the inductor 574 located in a middle portion 500a of the device 200, an inductive charging station for charging the device 500 will have to take a different form compared to that previously discussed. For example, the inductive charging station may sit next to or wrap around the middle portion 500a of the device, rather than interacting with the base.

FIG. 13b shows a schematic view of an inductive charging arrangement 600 used to charge the battery in the devices described herein. The charging arrangement 600 comprises two ferrite rods 601 (with a diameter of approximately 4 mm and a length of approximately 30 mm). One of the two rods 601 is mounted on a PCB of the transillumination device and the other is positioned in a separate inductive charging device. Each of the rods is surrounded (in part) by a copper coil 602a, 602b with a thickness of 0.25 mm, this forming an inductor in each of the handheld device and charging device. When assembled in the devices (the handheld transillumination device and the charging device respectively) a 2.5 mm tolerance gap is left between the outside of the coils 602a, 602b of the respective inductors to allow for walls of the respective devices and tolerance between them during charging.

During charging, the coils 602a, 602b are each part of a resonant circuit, the two resonant circuits are loosely magnetically coupled. The coil 602a is arranged to receive energy through inductive coupling with the coil 602b of the charging station. Alternating current passes through the induction coil 602b in the charging station. The moving electric charge from this creates a magnetic field, which fluctuates in strength because the electric current's amplitude is fluctuating. This changing magnetic field, enhanced by the rods 601, creates an alternating electric current in the coil 602a of the device, which in turn passes through a rectifier to convert it to direct current. The direct current is then used to charge the rechargeable battery of the transillumination device.

FIGS. 14a and 14b show the interior of a transillumination device 700. The device comprises a main body portion 701 including an outer housing 702 having a base portion 703, a middle portion 704 and a neck portion 705. On the neck portion there is mounted a head portion 800, in the form of a tip as previously described. Inside the housing 702 there can be seen a battery 707 arranged for inductive charging as described above, along with rigid printed circuit boards 708, 709 (PCBs) orthogonal to one another for holding various electronic components. This maximises the space available for mounting components.

In the neck portion 705, seen more clearly in FIGS. 14b and 14c, there is a transparent window 710 made of sapphire glass that allows the passage of light into the interior of the neck portion 705. Inside of the neck portion an optical assembly 711 can be seen. This window is resistant to scratching, which is important as it is exposed during use of the device, in a user's mouth. The optical assembly comprises a camera 712 that is positioned opposite (i.e. behind) the transparent window 710. Thus, the transparent window protects the optical assembly. The optical assembly also comprises a plastic housing 713 above the camera, an aperture film 714, a lens 715, a metal frame 716 for holding the transparent window 710 in place and a rubber gasket 717 for holding the optical assembly together. The camera 712 is mounted on a flexible PCB 718 within the neck portion 705 of the main body portion of the device. The camera 712 is also secured to the rigid PCB 709 (along with the other components of the optical assembly) to provide support and reduce relative movement of these components.

A heater (not shown) is arranged to heat the metal frame 716, which in turn conducts heat to the transparent window 710 and prevents fogging/condensation in use. To do so the window may be heated to 40° C. within 20 seconds, for example. In use, with a tip 800 mounted as shown, transillumination of a tooth is performed and the camera 712 captures images through the transparent window 710. In use the transparent window 710 is exposed to the conditions (heat and humidity) of a user's month.

FIGS. 15a to 15c show the internal arrangement of PCBs 708, 709 and electronic components more clearly. FIG. 15a shows a first rigid PCB 708 and a second rigid PCB 709. These are oriented substantially at a right angle to one another to allow maximum space for mounting electronic components inside of the housing. The first PCB 708 has an antenna 719 (positioned in the base of the device) and a cradle 720 mounted upon it; the cradle holds the battery 707. The second PCB 709 has a connection point 721 mounted on it, in the neck portion of the device. The connection point 721 is electrically connected to a pogo connector that extends through the housing of the main body to which a corresponding pogo connector of a head portion can be connected. When the tip 800 is mounted on the neck portion (as shown in FIG. 14c) this connection point 721 therefore provides an electrical connection point between the main body portion of the device and the head portion. This makes is easy for the tip to be mounted and removed from the device, for example to swap it with another. Also attached to the second PCB 209 is a flexible PCB 718 on which the optical assembly 711 is mounted.

FIGS. 16a and 16b show the tip arrangement in more detail. The flexible PCB 801 in the tip extends into first and second flexible wing portions 802, 803 extending from a central portion of the tip. On each of these portions are mounted LEDs 804 for illuminating a tooth and performing the transillumination method previously described. The optical assembly 711 within the neck portion of the device can also be seen in this figure. The transparent window 710 of the optical assembly aligns with an aperture 805 in the tip.

FIG. 16b shows an exploded view of the tip portion 800, including the flexible PCB 801, and a flexible over-moulding 806 which encapsulates the flexible PCB 801.

It will be appreciated that the devices and methods described herein could be adapted for use on the teeth of animals. In such a case, the device would be more robust that a device made for human-use (comprising a stronger casing to withstand chewing by an animal, for example). The user of the device would then be a veterinary professional or possibly the animal's owner. Different sized/shaped head portions 110 for the transillumination device 100 would also be provided, with a size and shape appropriate for use with different animals—for example, dogs, cats and horses. The LEDs 122 and lens/prism arrangements could also be adapted accordingly.

1. A head portion for a device for transillumination of a tooth, the head portion comprising:
a central portion, arranged to receive light from the tooth for imaging an occlusal/incisal view of the tooth, and
first and second flexible wing portions extending from the central portion, wherein the first flexible wing portion is configured to contact a first side of the tooth, and the second flexible wing portion is configured to contact a second side of the tooth, opposite to the first side,
wherein the first flexible wing portion comprises a first plurality of illumination sources for illuminating the tooth from the first side, and the second flexible wing portion comprises a second plurality of illumination sources for illuminating the tooth from the second side, and wherein the first and second plurality of illumination sources are controllable to produce multiple different lighting conditions.

2. A head portion as claimed in claim 1, wherein the illumination sources of the first and second plurality of illumination sources are independently controllable, or controllable in pairs, or controllable in groups of greater than two.

3. A head portion as claimed in claim 1 or 2, wherein the plurality of illumination sources are LEDs.

4. A head portion as claimed in any preceding claim, wherein the plurality of illumination sources are configured to emit near-IR light.

5. A head portion as claimed in any preceding claim, wherein the first plurality of illumination sources comprises four illumination sources, and the second plurality of illumination sources comprises four illumination sources.

6. A head portion as claimed in claim 5, wherein the four illumination sources of the first and/or second plurality of illumination sources are arranged at the corners of a notional square, rectangle, rhombus or parallelogram facing the side of the tooth.

7. A head portion as claimed in claim 5 or 6, wherein two of the four illumination sources are located so as to be positioned closer to the tip of the tooth when the head portion is in place on a tooth, and form a first group, and two of the four illumination sources are located so as to be positioned further from the tip of the tooth when the head portion is in place on a tooth, and form a second group.

8. A head portion as claimed in claim 7, wherein the first group and second group are controllable independently of each other.

9. A head portion as claimed in any preceding claim, wherein the intensity of illumination and/or the on/off status of the illumination sources is controllable.

10. A head portion as claimed in any preceding claim, wherein the illumination sources are controllable in such a way as to illuminate the first and second sides of the tooth simultaneously and are controllable to illuminate only one of the first and second sides at a time.

11. A head portion as claimed in any preceding claim, wherein each of the first and second flexible wing portions comprise a projection arranged to space apart the illumination sources from the tooth.

12. A head portion as claimed in any preceding claim, wherein each of the first and second flexible wing portions comprise a projection arranged to prevent or reduce light from the illumination sources directly falling into a window in the central portion.

13. A head portion as claimed in any preceding claim, wherein the central portion comprises a plurality of alignment projections bracketing the first and second flexible wing portions and arranged substantially perpendicular to the first and second flexible wing portions.

14. A head portion as claimed in any preceding claim, comprising a flex PCB on which the plurality of illumination sources are mounted and/or a comprising a chip for controlling the illumination sources.

15. A head portion as claimed in any preceding claim, comprising a RFID tag or NFC tag.

16. A transillumination device comprising the head portion of any preceding claim, wherein the head portion is removably attached to the transillumination device.

17. A transillumination device according to claim 16, wherein the transillumination device comprises:
an optical assembly and a camera, wherein the optical assembly guides light received through a window in the head portion to the camera;
and/or a controller for controlling the illumination sources;
and/or a wireless transceiver.

18. A method of imaging a tooth by transillumination comprising:
controlling a plurality of illumination sources to apply a first lighting condition to the tooth, and imaging the tooth under the first lighting condition to acquire a first image;
controlling the plurality of illumination sources to apply a second lighting condition to the tooth, and imaging the tooth under the second lighting condition to acquire a second image,
wherein the second lighting condition is different to the first lighting condition.

19. A method according to claim 18, comprising analysing the first and second images to determine if the first and second images meet predetermined criteria for further analysis.

20. A method of imaging a tooth by transillumination comprising:
controlling a plurality of illumination sources to apply a first lighting condition to the tooth, wherein the first lighting condition is applied on the basis of knowledge of the type of tooth that is being imaged, and imaging the tooth under the first lighting condition to acquire a first image.

21. A method according to claim 20, comprising analysing the first image to determine if the first image meets predetermined criteria for further analysis, and if not, controlling a plurality of illumination sources to apply a second lighting condition to the tooth, and imaging the tooth under the second lighting condition, wherein the second lighting condition is different to the first lighting condition.

22. A method of imaging a tooth by transillumination comprising:
controlling a plurality of illumination sources to apply a first lighting condition to the tooth, wherein the first lighting condition is pre-set based on a prior calibration process carried out by the user; and
imaging the tooth under the first lighting condition to acquire a first image.

23. A method according to claim 22, wherein the prior calibration process comprises:
sequentially applying a plurality of lighting conditions to each tooth;
acquiring an image of each tooth under each lighting condition;
for each tooth, analysing the images taken under each lighting condition, and determining the highest quality image;
for each tooth, setting the lighting condition under which the highest quality image was acquired as the first lighting condition.

24. A method according to any of claims 18 to 23, comprising use of the head portion of any of claims 1 to 15 or the transillumination device of claim 16 or 17.

25. A method according to claim 24, comprising imaging the tooth while the head portion is held stationary on the tooth, or whilst the head portion is being moved across the surface of the tooth.

26. A method according to any of claims 18 to 25, comprising controlling the plurality of illumination sources individually, in pairs, or in groups of greater than two.

27. A method according to any of claims 18 to 26, wherein the plurality of illumination sources are LEDs, which optionally emit near-IR light.

28. A method according to any of claims 18 to 27, comprising controlling the intensity of illumination and/or the on/off status of the illumination sources.

29. A method according to any of claims 18 to 28, comprising controlling the illumination sources to illuminate a first side and a second side of the tooth simultaneously.
